(19) United States
(12) Patent Application Publication
(10) Pub. No.: US 2024/0024133 A1
(43) Pub. Date: Jan. 25, 2024

(54) METHOD AND COMPUTER PROGRAM FOR CREATING MANUFACTURING DATA, AND METHOD FOR MANUFACTURING AN ORTHOPEDIC DEVICE

(71) Applicant: OTTOBOCK SE & CO. KGAA, Duderstadt (DE)

(72) Inventors: Michael SCHMIDT, Göttingen (DE); Christian KÄPPEL, Berlin (DE); Christian DAUR, Berlin (DE)

(21) Appl. No.: 18/247,982

(22) PCT Filed: Oct. 4, 2021
(86) PCT No.: PCT/EP2021/077288

§ 371 Date: Oct. 10, 2023

(30) Foreign Application Priority Data
Oct. 8, 2020 (DE) 10 2020 126 435.6

(51) Int. Cl. A61F 2/50; B29C 64/393; B33Y 80/00; B33Y 50/02

(52) U.S. Cl. CPC A61F 2/5046; B29C 64/393; B33Y 80/00; B33Y 50/02; A61F 2002/505; A61F 2240/002

(57) ABSTRACT

The invention relates to a method for creating manufacturing data for manufacturing an orthopedic device, which is producible using the created manufacturing data in an automated manufacturing method, wherein the method comprises the following steps:
providing a digital 3D body part model of a body part in a data processing facility,
providing at least one digital functional component model of an orthopedic functional component in the data processing facility, which is integratable in an orthopedic device, wherein the digital functional component model contains corresponding component properties of the respective orthopedic functional component,
generating at least one digital component interface by means of the data processing facility in dependence on at least one component property of at least one selected digital functional component model of an orthopedic functional component, which is to be integrated into the orthopedic device, wherein the digital component interface includes a receptacle for arranging the at least one selected orthopedic functional component,
automatically creating a digital orthopedic model of the orthopedic device to be manufactured based on the 3D body part model and the digital component interface for integration of the orthopedic functional component by means of the data processing facility, and
generating the digital manufacturing data from the created digital orthopedic model by means of the data processing facility.

The invention relates to a method for creating manufacturing data for manufacturing an orthopedic device, which is producible in an automated manufacturing method using the created manufacturing data. The invention also relates to a computer program for this purpose.

The invention additionally relates to a method for manufacturing an orthopedic device based on the created manufacturing data.

Orthopedic devices, such as orthoses or prostheses or also exoskeletons, have to be developed and matched precisely and accurately for the prevailing conditions of the handicapped person or the person to be assisted. A large number of manual work steps to be executed by hand are also still necessary for this purpose today, in order to produce such an orthopedic device.

In practice, for this purpose initially a negative impression of a body part, for which the orthopedic device is intended, is created from a molding compound. The negative impression is subsequently cast, for example using plaster. This body part model thus created is generally adapted or slightly changed by an orthopedic technician knowing a known medical indication or a desired form of assistance, so that the later orthopedic device, such as an orthosis or also a prosthesis, can fulfill its medical purpose or its provided form of assistance. The model of the relevant body part thus adapted to the medical indication is also called an orthopedic intended shape.

Based on the intended shape thus created, the orthopedic device is now developed and produced for the handicapped person or person to be assisted, in that the orthopedic device is adapted with its shape, geometry, and dimensions to the created intended shape.

It is disadvantageous in this case that such a process for producing such an orthopedic device is work intensive and usually requires several days until the orthopedic device can be presented to the handicapped person. The reason for this is the fact that multiple different groups of persons participate in the entire process, who each have different tasks in the creation of the orthopedic device. Adaptations which are required in the course of the process thus require a long time until the relevant person or person group can assume the task. Moreover, the process is strongly dependent on the experience of the orthopedic technician and supplies different results in multiple passes.

A method for creating manufacturing data for automatically producing an orthopedic device by means of an automated manufacturing facility is known from 10 2019 109 781.9 (published subsequently), in which initially a digital intended shape is provided.

Based on the digital intended shape, a volume model of the orthopedic device to be produced is created by means of a data processing facility, wherein the manufacturing data for the automated manufacturing facility are then generated based on the digital volume model.

Depending on the intended use of the orthopedic device, complex mechanical and/or mechatronic components such as hydraulics, circuits, batteries, processors, actuators, sensors, etc. have to be integrated into the orthopedic device in order to provide the orthopedic device with additional functions. These components designated as functional components generally have a complex structure and also cannot be produced or printed in an economically reasonable manner in an automated manufacturing method within a foreseeable time.

Therefore, the later integration of one or more functional components has to be taken into consideration in the design process during the creation of an orthopedic device, which slows the production process as such and makes it significantly more cost-intensive. This is because not only the simple integration of the functional component is decisive here, but rather also the interplay and cooperation of the functional component with the orthopedic device during the intended use. It thus has to be ensured, for example, that the integrated functional component has sufficient movement space to be able to execute its function and in addition is integrated into the orthopedic device so that the occurring loads can be dissipated and do not result in damage of the orthopedic device. Such an orthopedic device can make up a majority of the later overall system (device plus functional component) or also the orthopedic device only plays a subordinate role (for example solely of a cosmetic nature), in which larger functional components are integrated (such as knee joints).

It is therefore the object of the present invention to specify an improved method for producing an orthopedic device, using which components may also be integrated with a reliable process.

The object is achieved by the method for creating manufacturing data as claimed in claim 1, the computer program as claimed in claim 14, and the method for manufacturing an orthopedic device as claimed in claim 15. Advantageous embodiments of the invention are found in the corresponding dependent claims.

As claimed in claim 1, a method is proposed for creating manufacturing data for manufacturing an orthopedic device, wherein the orthopedic device is producible or is to be produced using the created manufacturing data in an automated manufacturing method. The manufacturing data are accordingly used as the foundation for activating an automated manufacturing facility, which executes the automated manufacturing method for producing the orthopedic device. Such manufacturing data can be, for example, computer models, on the basis of which control signals for activating the automated manufacturing facility for producing the orthopedic device are generated. The manufacturing data can also already contain such control signals or consist of such control signals, however. For example, the manufacturing data can also be a computer model which is then loaded into so-called slicer software, which divides the model into individual layers for activating a 3D printer.

An automated manufacturing method using an automated manufacturing facility is understood in particular as a method in which the orthopedic device is produced without human comprehension being interposed and substantially without manual intervention. Such an automated manufacturing method can be, for example, an additive or generative manufacturing method, such as a 3D printing method. Subtractive methods such as CNC milling are also conceivable, however. Such automated manufacturing methods can also be summarized here under the term “rapid manufacturing process”.

An orthopedic device in the meaning of the present invention can be, as already briefly indicated above, an orthosis or prosthesis or an orthosis part or prosthesis part. An orthosis as an orthopedic device can be in this case, for example, a foot orthosis, hand orthosis, knee orthosis, torso orthosis, or head orthosis. A prosthesis as an orthopedic device can also be in this case, for example, a knee prosthesis, arm prosthesis, a prosthesis socket, a cosmetic prosthesis, a prosthetic foot, or a prosthetic hand. However, this list is not to be understood as exhaustive.

Exoskeletons, which are arranged externally on a body part or the entire body of the wearer and are to enable movements and/or activities that can no longer be carried out by the body itself or are to assist the wearer in movements or activities, are orthopedic devices in the meaning of this invention. This also includes devices which make it easier for the wearer to carry out strenuous, exhausting, or fatiguing activities, for example overhead work, better, more easily, and for a longer time.

It is now provided according to the invention that initially a digital 3D body part model of a body part is provided in a data processing facility. Furthermore, at least one digital functional component model is provided in the data processing facility, wherein the digital functional component model digitally depicts an orthopedic functional component, which is integratable in an orthopedic device. Each of the digital functional component models contains corresponding component properties of the respective orthopedic functional component, so that the orthopedic functional components are entirely or partially described by their respective functional component models and the component properties contained therein. An orthopedic functional component expands the functional scope of the orthopedic device here and can provide specific functions to the wearer of the orthopedic device. However, the functional component can also first provide the function or the entire functionality, for example, as in the case of a cosmetic prosthesis as an orthopedic device which first provides the function in conjunction with a knee joint having a foot. It is conceivable and also advantageous here if a plurality of digital functional component models are each provided for different orthopedic functional components. One or more functional component models can be provided here for an orthopedic device.

A digital component interface is now generated by means of the data processing facility, which is provided to integrate a selected functional component on or in the orthopedic device. The digital component interface is generated here in dependence on component properties of at least one selected digital functional component model of an orthopedic functional component, which is to be integrated into the orthopedic device, wherein the digital component interface includes a receptacle for arranging the at least one selected orthopedic functional component. The receptacle can include, for example, recesses, fastening devices, and/or supports, which are capable of establishing a connection between the orthopedic device and the respective functional component.

Subsequently, by means of the data processing facility, a digital orthopedic model of the orthopedic device to be manufactured is generated based on the 3D body part model and the digital component interface for integrating the orthopedic functional component, by which the orthopedic device to be produced is digitally defined enough that all properties are present except for the integration of the respective functional component. Of course, it is also possible that a user, both before and after the generation of the digital orthopedic model, performs adjustments thereon or the underlying data in a manual or automated manner.

Finally, the digital manufacturing data can be generated from the created digital orthopedic model by means of the data processing facility, in order to thus produce the orthopedic device in an automated manner with supply of the digital manufacturing data to a digital manufacturing facility, so that subsequently only the functional components still need to be integrated by means of the component interface and its receptacle.

It is thus possible to produce orthopedic devices in a substantially automated manner, wherein functional components which have to or are supposed to be integrated into the orthopedic device can be integrated seamlessly into the produced orthopedic device at a later point in time. By way of the definition of the functional components in the form of digital functional component models, all properties of these functional components can be described, so that these properties of the functional components can be taken into consideration in the creation of the digital component interface.

The created digital orthopedic model is in this case in particular a digital 3-dimensional model, which contains or describes the shape, geometry, dimensions, and properties of the orthopedic device to be produced.

The provided 3D body part model can be, for example, a model of a body part to which an orthopedic device is to be attached (for example an orthosis or a prosthesis). The 3D body part model can also, however, be a model of a contralateral body part, which is used as the foundation for the simulation of the respective other body part side. This is the case, for example, if a contralateral body part which is still present is to be simulated by means of a cosmetic prosthesis.

According to one embodiment, it is provided that the 3D body part model is a digital depiction of the body part, for which the orthopedic device is intended, which was converted into an orthopedic intended shape. The 3D body part model can thus not only be a digital depiction of the body part, for which the orthopedic device is intended, but can also contain or represent an orthopedic intended shape, which is indicated by a medical indication and/or takes into consideration individual anatomic conditions. In addition, the intended shape offers the possibility for the orthopedic technician of performing their own adaptations to the depiction and thus having their own experiential values incorporated. The socket can thus be shaped, for example, so that bony structures and soft parts get different pressure loads.

The digital depiction can be produced, for example, by a digital scan or by an imprint by means of a molding compound. A parametric acquisition of the body part is also conceivable, in which the orthopedic technician measures important dimensions of the body part and creates the body part model by means of the data processing facility. However, methods such as stereometry or MRT/CT are alternatively also conceivable. It is also conceivable to use a depiction of the contralateral body part. This is advantageous in particular in the creation of prostheses or cosmetic prostheses since they can thus be adapted to the appearance of the contralateral side still present. The use of generic body part models from databases or simulations is also conceivable. These can be adapted to the patient as needed, for example via parameters such as height and weight.

According to one embodiment, it is provided that the provided 3D body part model was freely modeled or can be freely modeled by the orthopedic technician. The modeling can take place digitally or also initially by means of plaster or other aids and can subsequently be scanned. In this way, the orthopedic technician can model the body part model even independently of the actual appearance. This procedure can be advantageous in particular in cosmetic prostheses, since the appearance of the cosmetic can thus be individually adapted and can also deviate if desired from the original body part.

According to one embodiment, it is provided that a digital orthopedic model is created in the form of a volume model. A volume model has the advantage in relation to a two-dimensional model that the wall thickness of the orthopedic device is visible.

According to one embodiment, it is provided that, to generate a digital mechanical interface, a digital mechanical interface is selected from a plurality of provided digital mechanical interfaces in dependence on component properties of the at least one selected digital functional component model of the orthopedic device, the receptacle of which corresponds to the at least one selected orthopedic functional component. It is accordingly additionally provided that initially a large number of digital mechanical interfaces are provided, from which that mechanical interface is then selected which matches the selected functional component based on the component properties. The mechanical interface and also the functional component are therefore selected from a library of already prefinished mechanical interfaces, so that more or less standard interfaces can be used.

According to one embodiment, it is provided that the digital component interface is furthermore generated in dependence on the 3D body part model and/or on a digital model, created from the 3D body part model, of the orthopedic device to be manufactured.

According to one embodiment, it is provided that a first digital orthopedic partial model of the orthopedic device to be manufactured is automatically created based on the 3D body part model by means of the data processing facility and a second digital orthopedic partial model of the orthopedic device to be manufactured is created based on the generated digital component interface, wherein the digital orthopedic model of the orthopedic device to be manufactured is created in dependence on the first digital orthopedic model and the second digital orthopedic model.

Accordingly, at least two orthopedic partial models are created, which are adapted to the 3D body part model, on the one hand, and depict the mechanical interface, on the other hand, wherein the two partial models are then joined together upon the creation of the digital orthopedic model, in order to generate a common model in which the mechanical interface is integrated in the orthopedic device. The creation of the second digital orthopedic partial model therefore not only comprises the provision of the receptacle for the functional component, but can also comprise a further receptacle, using which the mechanical interface is arranged on the first partial model in order to obtain the complete model. This second receptacle is dependent on the first partial model in this case.

It is therefore particularly advantageous if the second digital orthopedic model of the orthopedic device to be manufactured is furthermore created in dependence on the previously created first digital orthopedic model.

According to one embodiment, it is provided that for each digital functional component model of an orthopedic functional component, as a component property, the dimensions of the component, an installation space of the component, a movement space of the component, stability properties, accesses for the installation and/or removal, tool attachments, supply lines, disposal lines, tolerable torques, occurring torques, possible functional component combinations, and/or thermal properties are stored. Functional component combinations are necessary insofar as more than one functional component is to be integrated, since not every functional component also technically harmonizes with every other functional component. By storing a matrix of functional component combinations, it becomes possible to make available only reasonable combinations.

According to one embodiment, it is provided that one or more component properties are visualized on a playback unit of the data processing facility with or without the digital orthopedic model or a partial model thereof. By visualizing the component properties, for example, movements of the functional component can be represented, by which it becomes apparent whether the generated orthopedic model meets the boundary conditions for the function of the functional component.

According to one embodiment, it is provided that in dependence on at least one component property of at least one selected functional component, it is checked by means of the data processing facility whether the combination of selected functional component and provided 3D body part model, an orthopedic intended shape derived therefrom, and/or digital orthopedic model or partial model is producible and/or functional. Functional in this case means in particular that the orthopedic device together with the functional component as an overall system can carry out the desired movements and/or withstand the expected loads.

In this way, it additionally becomes possible to have it checked automatically by the data processing facility whether the combination of selected functional components and the body part model, an intended shape derived therefrom, or the already created orthopedic model or partial model is producible or technically functions at all and the provided functions of the functional components can be implemented. It may thus be checked whether the movement space is present so that the movement specified by a functional component is executable. It may also be checked whether accesses are selected so that technicians have access to supply lines or other functional components. Moreover, it may be checked whether the expected loads can be withstood and/or dissipated.

According to one embodiment, it is provided that the movement and/or the load of the orthopedic device to be produced and/or the selected functional component are simulated in dependence on at least one component property of at least one selected functional component by means of the data processing facility. The functions of the integrated functional components can be digitally tested here by an extensive simulation.

According to one embodiment, it is provided that a digital surface model of the orthopedic device to be manufactured is created, based on the 3D body part model and a border of the orthopedic device specified on the 3D body part model, by means of the data processing facility, wherein the surface model forms the inside of the later orthopedic device and wherein the digital orthopedic model of the orthopedic device to be manufactured is automatically created based on the digital surface model and a specified material thickness of the orthopedic device to be manufactured by means of the data processing facility.

According to one embodiment, it is provided that the orthopedic device is an orthosis, in particular a foot orthosis, hand orthosis, knee orthosis, torso orthosis, or head orthosis, a prosthesis, or an exoskeleton.

The object is moreover also achieved by the computer program as claimed in claim 14 for carrying out the above-mentioned method for creating manufacturing data when the computer program is executed on a data processing facility. The computer program can advantageously be stored on a data carrier here.

The object is moreover also achieved according to the invention by the method for manufacturing an orthopedic device as claimed in claim 15, wherein initially the manufacturing data for the orthopedic device are created using the method as described above. Subsequently, these manufacturing data are supplied to an automated manufacturing facility, which is configured to produce the orthopedic device using the created manufacturing data in an automated manufacturing method. Such a manufacturing facility can be, for example, a 3D printer. Such manufacturing facilities are known, for example, under the concept of additive or generative manufacturing facilities. By supplying the manufacturing data to the automated manufacturing facility, the orthopedic device is then produced by the automated manufacturing method in dependence on supplied manufacturing data.

The described data processing facility can be a single device, which is operated by one or more persons simultaneously or in succession. However, it is also conceivable that the data processing facility is distributed over multiple computer systems, so that here, for example, multiple persons have access at different locations. It is thus conceivable that the creation of the 3D body part model is generated by a first part of the data processing facility, while the creation of the orthopedic model is carried out by a second part of the data processing facility. The generation of the manufacturing data can then be carried out, for example, by a third part of the data processing facility. The first, second, and third part of the data processing facility do not necessarily have to be depicted by the same device here but can be implemented at various locations by independent separate devices (computing units) in each case.

The invention will be explained in more detail by way of example on the basis of the appended figures. In the figures:

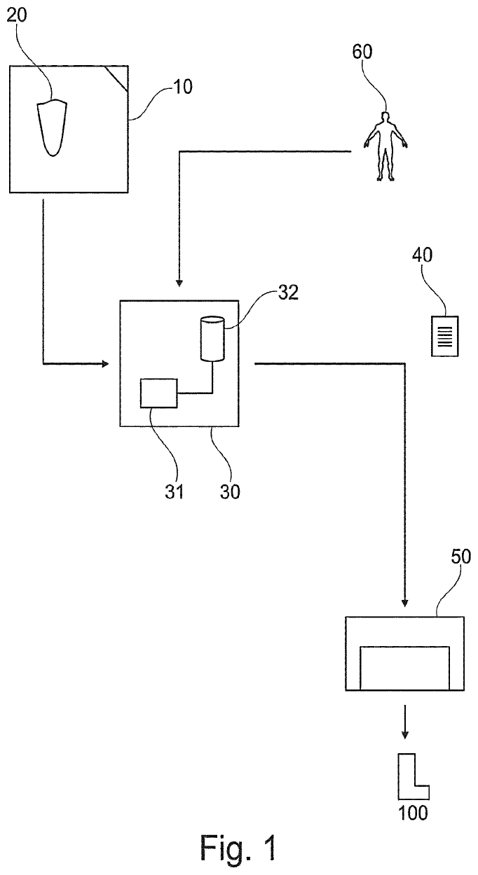

FIG. 1 shows a schematic representation of the method sequence according to the invention;

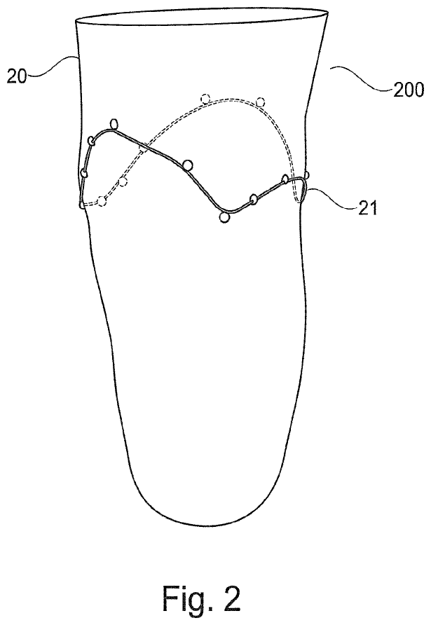

FIG. 2 shows a representation of a 3D body part model;

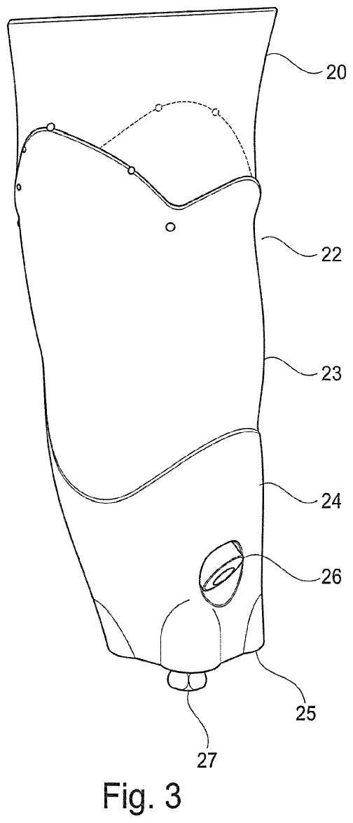

FIG. 3 shows a representation of an orthopedic 3D model;

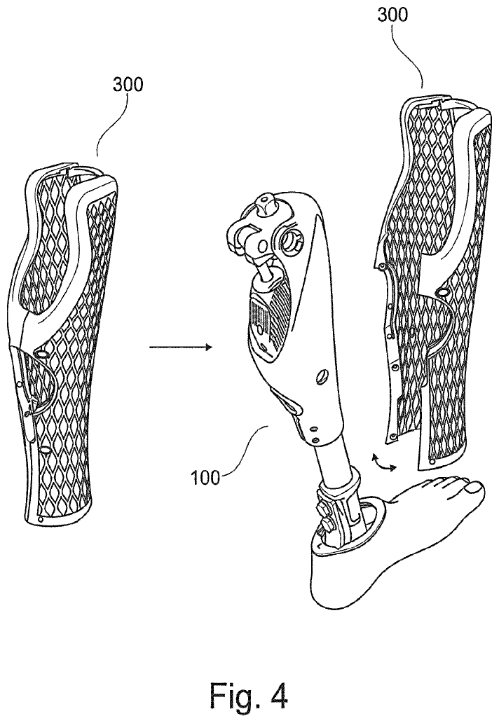

FIG. 4 shows a representation of a cosmetic prosthesis during use;

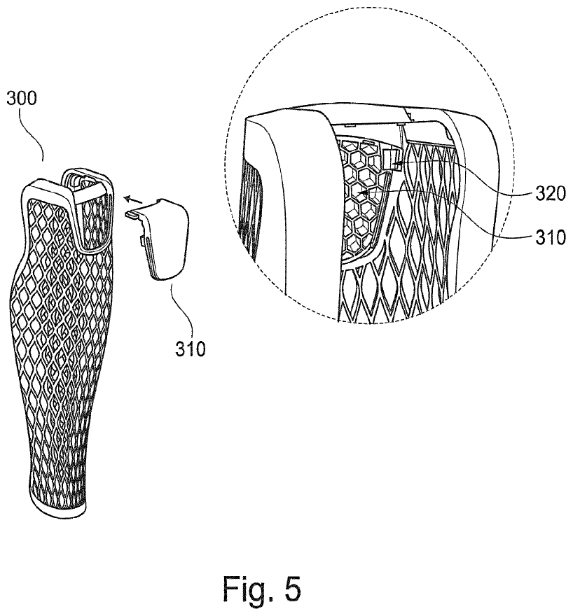

FIG. 5 shows a representation of the integration of a first functional element;

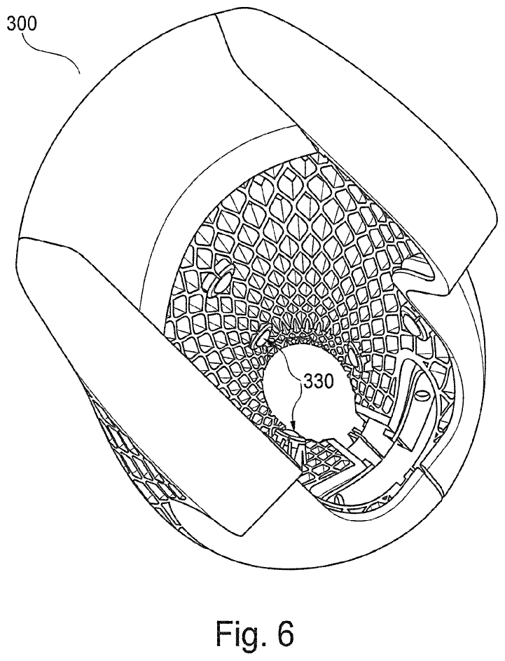

FIG. 6 shows a representation of an interior of a cosmetic prosthesis;

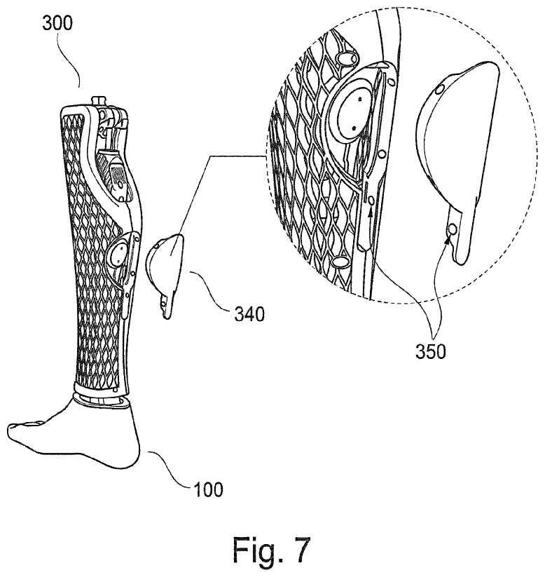

FIG. 7 shows a representation of an integration of a second functional element.

FIG. 1 shows a schematic representation of the method sequence according to the invention, which begins with the provision of a 3D body part model 20 in the form of a digital intended shape 10. The 3D body part model 20 is a three-dimensional depiction of the relevant body part of the handicapped person. In the exemplary embodiment of FIG. 1, the digital intended shape 10 is an amputation stump of an amputated leg, for which a prosthesis is to be manufactured.

In the ideal case, the digital intended shape already has a shape and geometry, using which the medical indication of the handicapped person is to be treated as a measure. The digital intended shape accordingly has a shape and geometry which then results in a corresponding shape and geometry of the orthosis, using which the medical indication of the handicapped person may be treated and therefore represents the suitable treatment measures. Alternatively, the digital intended shape has a shape adapted to the individual anatomy of the handicapped person, which then results in a corresponding shape and geometry of the orthosis or prosthesis, using which this is matched particularly well to the shape of the body part and therefore results in increased wearing comfort. In particular in the case of prosthesis sockets, it is regularly advantageous to modify the 3D body part model and thus create an intended shape which takes into consideration the distribution of soft tissue, muscles, and bones.

The digital intended shape 10 having the 3D body part model 20 is now provided to a data processing facility 30, which has a computing unit 31 and a data memory 32. Computing unit and data memory can also be cloud-based solutions here. A plurality of digital functional component models are provided in the data memory 32, which are each integratable into the orthopedic device to be produced and create or provide a special additional function for the orthopedic device.

In the first step, the computing unit 31 in the exemplary embodiment of FIG. 1 is designed so that, based on the 3D body part model 20 or the digital intended shape 10, it carries out an analysis to thus create a first digital orthopedic model of the orthopedic device to be manufactured.

A technician 60 can influence the first partial model here. The technician 60 furthermore has the option of selecting one or more orthopedic functional components which are to be integrated into the orthopedic device to be produced. Based on the selection of the technician 60, the digital functional component models are then retrieved from the data memory 32, including the component properties stored for each digital functional component model.

In the next step, the computing unit 31 in the exemplary embodiment of FIG. 1 is designed so that based on the component properties of at least one selected digital functional component model of an orthopedic functional component, a digital component interface is generated, from which a second digital orthopedic partial model is then created. The digital component interface includes a receptacle here, which is adapted individually to the selected functional component based on the component properties and the digital functional component model so that the functional component may be integrated without problems into the digital component interface.

In the area of the receptacle, the digital component interface is adapted here to the selected functional component in accordance with the component properties in order to be able to receive the functional component later without problems.

A digital orthopedic model of the orthopedic device is now created by means of the computing unit 31 from the first digital orthopedic partial model and the second digital orthopedic partial model in that the two partial models are combined to form a joint overall model. The digital component interface is adapted here in the area in which the second partial model is fused with the first partial model, preferably so that the second partial model essentially corresponds to the first partial model in this area.

After the creation of the digital orthopedic model, corresponding manufacturing data are then generated which are then transferred to an automated manufacturing facility 50. The manufacturing data 40 can in the simplest case involve the created digital model, which is then analyzed by the automated manufacturing facility 50 to activate the facility, in order to generate the corresponding control signals for activating the automated manufacturing facility 50. However, it is also conceivable that the manufacturing data 40 already contain those control signals which are used to activate the automated manufacturing facility 50. This is ultimately dependent on the specific application and the type of the automated manufacturing facility 50 or the automated manufacturing method executed by the manufacturing facility 50.

In the exemplary embodiment of FIG. 1, the automated manufacturing facility 50 is a 3D printing facility, using which the prosthesis 100 can be automatically produced based on the digital model in an additive or generative manufacturing method. After the production of the prosthesis 100, the receptacles of the respective functional component provided in the component interface can then be physically inserted into the prosthesis 100. The prosthesis 100 can in this case, for example, be a prosthesis socket, on which additional prosthesis elements still have to be arranged.

The data processing facility 30 is additionally configured to allow a manual intervention by a technician 60, in order to thus create the option of manually manipulating the model and thus the orthopedic device to be produced. In this way, special wishes can be taken into consideration, which are not producible in an automated manner.

FIG. 2 shows an amputation stump 200 of an amputated leg, on which a prosthesis is to be arranged. The amputation stump 200 shown in FIG. 2 is a 3D body part model 20 here, which is the foundation for the creation of the prosthesis socket. The 3D body part model 20 was already supplemented by a technician here in such a way that borders 21 are provided, which represent the upper terminus of the prosthesis socket.

FIG. 3 shows the finished digital orthopedic model 22, as it is arranged on the 3D body part model 20. The digital model 22 includes a first digital partial model 23 and a second digital partial model 24 here, which are fused to form a common digital model 22. The second digital partial model 24 forms the digital component interface using which functional components 26, 27 are to be integrated into the orthopedic device as a whole.

This digital model 22 can now be used as the foundation for generating digital manufacturing data, in order to thus have an orthopedic device then produced on this basis, which can then be inserted later into the provided receptacles corresponding to the functional component.

FIG. 4 shows a cosmetic prosthesis 300 as an orthopedic device, wherein the cosmetic prosthesis 300 is to be arranged on a mechatronic knee joint 100 as a functional component. The functional component permits here, for example, controlled damping of the knee movement. Cosmetic prosthesis 300 and functional component 100 together form the prosthetic knee. A prosthetic foot is arranged distally on the prosthetic knee as a further functional component. The cosmetic prosthesis 300 can also provide an interface for this further functional component. The cosmetic prosthesis 300 is used here to improve the appearance of the prosthesis 100 and in particular to adapt the appearance to a contralateral body part which is still present, here a foot with a leg. Furthermore, the cosmetic prosthesis provides a protective effect for the functional components.

As shown in FIG. 5, further functional components 310 can also be integrated into such a cosmetic prosthesis 300, in order to further improve the function of the cosmetic prosthesis 300. In the exemplary embodiment of FIG. 5, a functional component 310 in the form of a soft knee is inserted here into the cosmetic prosthesis 300, in order not to damage the cosmetic prosthesis 300 when supporting or when kneeling down. The cosmetic prosthesis 300 includes an interface 320 for this purpose, in which the functional component 310 is inserted. In the exemplary embodiment of FIG. 5, the functional component 310 is inserted and held in a locking manner for this purpose.

During the generation of the digital component interface, upon selection of the functional component 310 shown in FIG. 5, a corresponding locking receptacle is to be provided in which the functional component 310 can be locked.

FIG. 6 shows a representation of a cosmetic prosthesis 300 known from FIGS. 4 and 5 with an inside view. A second component interface 330 is provided here in the interior of the cosmetic prosthesis 300, which forms a receptacle for arranging an orthopedic functional component in the form of a prosthesis. Due to the closing of the cosmetic prosthesis 300, this second interface 320 presses as elastic elements against the surrounding prosthesis (not shown), so that the cosmetic prosthesis 300 is thus mounted.

FIG. 7 shows a cosmetic prosthesis 300 in a further embodiment, in which a further, third functional component 340 is inserted or insertable. The functional component 340 shown in FIG. 7 is a cover, using which a functional unit located behind it and provided in the covered prosthesis is to be protected.

An opening is provided here in the cosmetic prosthesis 300, into which magnets are introduced in the border area, which form a third interface 350 together with the opening, in order to thus be able to accommodate the third functional component 340 (cover). Magnets are also introduced here into the third functional component 340, which interact with the magnets in the cosmetic prosthesis 300 so that the third functional component 340 is held magnetically in the opening. The cover ensures the protection of the functional component here, but also enables rapid and easy access thereto. In the illustrated example, easy access to the charging terminal of the functional component is thus enabled.

Further aspects of the cosmetic, such as the honeycomb structure, can also be adapted to the need of the respective selected functional components. A structure having many large openings can be provided, for example, for hydraulic knee joints, in order to thus enable a sufficient removal of the heat arising during damping.

### LIST OF REFERENCE NUMERALS

10 digital intended shape
20 3D body part model
21 border
22 digital orthopedic model
23 first digital partial model
24 second digital partial model
25 digital component interface
26 functional component
27 functional component
30 data processing facility
31 computing unit
32 data memory
40 manufacturing data
50 manufacturing facility
100 prosthesis
200 amputation stump
300 cosmetic prosthesis
310 second functional component
320 first interface
330 second interface
340 third functional component
350 third interface

1. A method for creating manufacturing data for manufacturing an orthopedic device producible using the created manufacturing data in an automated manufacturing method, comprising:
providing a digital three dimensional (3D) body part model of a body part in a data processing facility,
providing at least one digital functional component model of an orthopedic functional component in the data processing facility, wherein the orthopedic functional component is integratable in an orthopedic device, wherein the at least one functional digital functional component model contains corresponding component properties of the respective orthopedic functional component,
generating at least one digital component interface in the data processing facility in dependence on at least one component property of at least one selected digital functional component model of at least one selected orthopedic functional component which is to be integrated into the orthopedic device, wherein the at least one digital component interface includes a receptacle for arranging the at least one selected orthopedic functional component,
automatically creating a digital orthopedic model of the orthopedic device to be manufactured based on the 3D body part model and the at least one digital component interface for integration of the orthopedic functional component by the data processing facility, and
generating digital manufacturing data from the created digital orthopedic model automatically created by the data processing facility.

2. The method as claimed in claim 1, wherein the 3D body part model is a digital representation of the body part for which the orthopedic device is intended and which was converted into an orthopedic intended shape.

3. The method as claimed in claim 1 wherein a digital orthopedic model is created in a form of a volume model.

4. The method as claimed in claim 1 further comprising selecting a digital mechanical interface from a plurality of provided digital mechanical interfaces in dependence on component properties of the at least one selected digital functional component model of the orthopedic device, wherein a receptacle of the selected digital mechanical interface corresponds to the at least one selected orthopedic functional component.

5. The method as claimed in claim 1 wherein the generated at least one digital component interface is also generated in dependence on the 3D body part model and/or on a digital model, created from the 3D body part model, of the orthopedic device to be manufactured.

6. The method as claimed in claim 1 further comprising, using the data processing facility, automatically
a first digital orthopedic partial model of the orthopedic device to be manufactured is created based on the 3D body part model, and
a second digital orthopedic partial model of the orthopedic device to be manufactured is created based on the generated digital component interface (25),
wherein the digital orthopedic model of the orthopedic device to be manufactured is created in dependence on the first digital orthopedic model and the second digital orthopedic model.

7. The method as claimed in claim 6, wherein the second digital orthopedic model of the orthopedic device to be manufactured is created in dependence on the previously created first digital orthopedic model.

8. The method as claimed in claim 1 wherein, for each digital functional component model of an orthopedic functional component, at least one of
dimensions of the component,
an installation space of the component,
a movement space of the component,
stability properties,
accesses for the installation and/or removal,
tool attachments,
supply lines,
disposal lines,
tolerable torques,
occurring torques,
possible functional component combinations, and
thermal properties,
are stored as a component property.

9. The method as claimed in claim 1 further comprising providing for visualizing one or more component properties on a playback unit of the data processing facility with or without the digital orthopedic model or a partial model thereof.

10. The method as claimed in claim 1 further comprising, in dependence on at least one component property of at least one selected functional component (26, 27), checking with the data processing facility whether a combination of the selected functional component and the provided 3D body part model, an orthopedic intended shape derived therefrom, and/or digital orthopedic model or partial model is producible and/or functional.

11. The method as claimed in claim 1 further comprising simulating by the dataprocessing facility, in dependence on at least one component property of at least one selected functional component, at least one movement and/or load of the orthopedic device to be produced and/or the selected functional component.

12. The method as claimed in claim 1 further comprising creating a digital surface model of the orthopedic device to be manufactured based on the 3D body part model and a border of the orthopedic device specified on the 3D body part model by the data processing facility, wherein the digital surface model forms an inside of the orthopedic device and wherein the digital orthopedic model of the orthopedic device to be manufactured is automatically created based on the digital surface model and a specified material thickness of the orthopedic device to be manufactured by the data processing facility.

13. The method as claimed in claim 1 wherein the orthopedic device producible using the created manufacturing data is an orthosis a prosthesis, or an exoskeleton.

14. A computer program having computer executable program code in a non-transient storage medium configured for carrying out a method as claimed in claim 1 when the computer program is executed on a data processing facility.

15. A method for manufacturing an orthopedic device, comprising:
creating manufacturing data for the orthopedic device using the method as claimed in claim 1,
supplying the manufacturing data to an automated manufacturing facility, wherein the automated manufacturing facility produces the orthopedic device using the created manufacturing data in an automated manufacturing method.

16. The method as claimed in claim 15, wherein
the orthopedic device produced by the automated manufacturing method of the automated manufacturing facility, is produced in dependence on the supplied manufacturing data.

17. The method as claimed in claim 15 further comprising fastening at least one functional component on a receptacle of a component interface of the orthopedic device.

18. The method of claim 13 wherein the orthosis is selected from the group consisting of a foot orthosis, a hand orthosis, a knee orthosis, a torso orthosis, and a head orthosis.
